(19) United States
(12) Patent Application Publication
(10) Pub. No.: US 2020/0279631 A1
(43) Pub. Date: Sep. 3, 2020

(54) BIOMETRIC SECURED MEDICAL CHECK IN

(71) Applicant: Alclear, LLC, New York, NY (US)

(72) Inventors: Marisa Bass, New York, NY (US); Joe Trelin, Seattle, WA (US)

(21) Appl. No.: 16/802,885

(22) Filed: Feb. 27, 2020

Related U.S. Application Data
(63) Continuation of application No. 62/812,352, filed on Mar. 1, 2019.
(60) Provisional application No. 62/825,628, filed on Mar. 28, 2019.

(51) Int. Cl. G16H 20/13; G06F 21/32; A61J 7/00; G16H 10/60; G06Q 40/08; G06Q 20/10

(52) U.S. Cl. CPC G16H 20/13; G06F 21/32; G06Q 20/102; G16H 10/60; G06Q 40/08; A61J 7/0084

(57) ABSTRACT

A system for biometric secured medical check in receives a digital representation of a biometric for a person, uses the digital representation of the biometric to retrieve identity information for the person, and provides the identity information to a medical service electronic device to check in the person for a medical service. In various implementations, the system may use the digital representation of the biometric to retrieve a medical record identifier for the person and facilitate access to a record by the medical service electronic device for the person stored by a medical records electronic device, process payment for the medical service using payment information stored in association with the identify information, receive the digital representation of the biometric from a check in electronic device and provide an acknowledgement based on a response received from the medical service electronic device to the check in electronic device, and so on.

### CROSS-REFERENCE TO RELATED APPLICATION(S)

This application is a nonprovisional patent application of and claims the benefit of U.S. Provisional Patent Application No. 62/812,352, filed Mar. 1, 2019 and titled “Biometric Secured Medical Check In,” and U.S. Provisional Patent Application No. 62/825,628, filed Mar. 28, 2019 and titled “Biometric Secured Medical Check In,” the disclosures of which are hereby incorporated herein by reference in their entireties.

### FIELD

The described embodiments relate generally to securing identity information using biometrics. More particularly, the present embodiments relate to facilitating medical check ins using identity information secured using biometrics.

### BACKGROUND

People use medical service providers to obtain a variety of different medical services. By way of one example, people frequently visit (whether by appointment or not) doctors' offices or hospitals for routine physicals, diagnosis of various medical issues, vaccinations, surgical procedures, medication prescriptions, and so on. By way of another example, people visit pharmacies to obtain various medical products, such as prescriptions and/or over-the-counter medications.

In many situations, medical service providers employ personnel to check in people for medical services. Such personnel may guide people through filling out various forms and/or other procedures to obtain identity information, such as one or more names, addresses, telephone numbers, social security numbers, patient identification numbers or other identifiers, insurance data, financial data, medical history, and so on. The personnel may use this identity information to identify the appointment and/or medical service for which the person is checking in, provide details that one or more medical practitioners will use for the medical service, charge insurance or payment accounts for the medical service and/or prepare to do such, guide the person to where the medical service will be provided, and so on.

### SUMMARY

The present disclosure relates to a system for biometric secured medical check in. The system may receive one or more digital representations of biometrics for a person, use the digital representation of the biometric to retrieve identity information for the person, and provide the identity information to a medical service electronic device to check in the person for a medical service. In some implementations, the system may use the digital representation of the biometric to retrieve a medical record identifier for the person and facilitate access to a medical record for the person stored by a medical records electronic device. In various implementations, the system may process payment for the medical service using payment information stored in association with the identity information. In a number of implementations, the system may receive the digital representation of the biometric from a check in electronic device and provide an acknowledgement based on a response received from the medical service electronic device to the check in electronic device.

In various embodiments, a system for biometric secured medical check in includes at least one non-transitory storage medium that stores instructions and at least one processor. The at least one processor executes the instructions to receive a digital representation of a biometric of a person, use the digital representation of the biometric to retrieve identity information for the person, provide the identity information to a medical service electronic device to check the person in for a medical service, use the digital representation of the biometric to retrieve a medical record identifier for the person, and use the medical record identifier to facilitate access by the medical service electronic device to a medical record for the person stored by a medical records electronic device.

In some examples, the at least one processor facilitates the access by providing the medical record identifier to the medical service electronic device. In other examples, the at least one processor facilitates the access by providing the medical record identifier to the medical records electronic device and providing a response from the medical records electronic device to the medical service electronic device.

In a number of examples, the medical record includes a vaccination list. In some examples, the medical record includes at least part of a medical history. In various examples, the medical record includes an allergy list. In a number of examples, the medical record includes a current medication list.

In some embodiments, a system for biometric secured medical check in includes at least one non-transitory storage medium that stores instructions and at least one processor. The at least one processor executes the instructions to receive a digital representation of a biometric of a person, use the digital representation of the biometric to retrieve identity information for the person, provide the identity information to a medical service electronic device to check the person in for a medical service, and process payment for the medical service using payment information stored in association with the identity information.

In various examples, the payment information includes insurance information for the person. In some implementations of such examples, the at least one processor processes the payment by submitting an insurance payment request using the insurance information. In a number of implementations of such examples, the at least one processor processes the payment by providing the insurance information to the medical service electronic device. In various implementations of such examples, the at least one processor determines a copay associated with the medical service and the insurance information and obtains a payment from the person for the copay.

In a number of examples, the payment information includes a financial account number. In some implementations of such examples, the at least one processor processes the payment by charging the financial account number. In various implementations of such examples, the at least one processor processes the payment by providing the financial account number to the medical service electronic device.

In a number of embodiments, a system for biometric secured medical check in includes at least one non-transitory storage medium that stores instructions and at least one processor. The at least one processor executes the instructions to receive a digital representation of a biometric of a person from a check in electronic device, use the digital representation of the biometric to retrieve identity information for the person, check the person in for a medical service by providing the identity information to a medical service electronic device, receive a response from the medical service electronic device, and provide an acknowledgment based on the response to the check in electronic device.

In some examples, the acknowledgement prompts for authorization to access a medical record for the person. In various examples, the acknowledgement includes an instruction regarding a location to report to receive the medical service. In a number of examples, the at least one processor determines the medical service electronic device to provide the identity information using location information provided via the check in electronic device. In some examples, the at least one processor determines the medical service electronic device to provide the identity information using a location of the check in electronic device.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example system for biometric secured medical check in.

FIG. 2 depicts a flow chart illustrating a first example method for biometric secured medical check in. This method may be performed by the system of FIG. 1.

FIG. 3 depicts a second example system for biometric secured medical check in.

FIG. 4 depicts a flow chart illustrating a second example method for biometric secured medical check in. This method may be performed by the systems of FIGS. 1 and/or 3.

FIG. 5 depicts a flow chart illustrating a third example method for biometric secured medical check in. This method may be performed by the systems of FIGS. 1 and/or 3.

FIG. 6A depicts a third example system for biometric secured medical check in.

FIG. 6B depicts the system of FIG. 6A upon check in.

FIG. 7 depicts a flow chart illustrating a third example method for biometric secured medical check in. This method may be performed by the systems of FIGS. 1, 3, and/or 6A and 6B.

FIG. 8A depicts a fourth example system for biometric secured medical check in.

FIG. 8B depicts the system of FIG. 8A upon check in.

FIG. 9 depicts a flow chart illustrating a fourth example method for biometric secured medical check in. This method may be performed by the systems of FIGS. 1, 3, and/or 8A and 8B.

FIG. 10A depicts a fifth example system for biometric secured medical check in.

FIG. 10B depicts the system of FIG. 10A as medications are provided.

FIG. 11 depicts a flow chart illustrating a fifth example method for biometric secured medical check in. This method may be performed by the systems of FIGS. 1, 3, and/or 10A and 10B.

FIG. 12A depicts a sixth example system for biometric secured medical check in.

FIG. 12B depicts the system of FIG. 12A upon vending.

FIG. 13 depicts a flow chart illustrating a fifth example method for biometric secured medical check in. This method may be performed by the systems of FIGS. 1, 3, and/or 12A and 12B.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, apparatuses, methods, and computer program products that embody various elements of the present disclosure. However, it should be understood that the described disclosure may be practiced in a variety of forms in addition to those described herein.

Typical check in procedures for medical services are often burdensome, time consuming, and highly inefficient. People may find it inconvenient to fill out check in forms. Such forms may be lengthy, particularly when standardized to cover as many different patient situations as possible, and people may be required to fill out information that they have previously provided to other medical service providers and/or do not have currently available (such as when people do not have a copy of their full medical history or medical list on them when checking in, cannot remember vaccination dates, and so on). These kinds of procedures also involve personnel to provide the forms and/or otherwise obtain the information from the people, interpret the provided information and/or otherwise enter such information into various electronic systems, match information to appointments and/or schedule medical services if there is no appointment, charge insurance and/or payment accounts, calculate copays, and a variety of other tasks. Various of these issues may result in delays, burdens, and/or other inefficiencies, as well as failure to obtain useful information (such as insurance coverage, copays, and/or other payments that may not be collectible later, allergies that may cause complications during the medical services like latex allergies, and so on).

The present disclosure may make check in procedures less burdensome, time consuming, and inefficient by storing identity information for people that may be retrieved upon check in. However, some implementations of such an approach may use a great deal of storage and/or other electronic components in situations where each medical service provider stores the information. Such implementations may still involve people providing a great deal of duplicate information to different medical service providers. In other implementations of such an approach, identity information could be stored in a centrally accessible location that different medical service providers could access. However, such a solution could make it difficult for people to ensure that they have control over access to their identity information.

In some implementations, one or more biometrics could be used to control access to identity information. In such an implementation, people could provide the biometric to enable access and retrieval of the identity information. This may allow the person to check in by providing the biometric without specifying additional information.

However, biometrically securing access to centrally stored identity information may present other issues. The system that uses the biometrics to centrally control access to identity information may be configured to expect inputs (such as one or more digital representations of biometrics, requests for specific identity information and/or attestations regarding specific identity information, and so on) in a particular format. This may be solved by using identical electronic devices at all medical service provider locations so that biometrics and requests involving such are all submitted in an expected way, but this is not particularly flexible.

However, the present disclosure may resolve such issues by using client apps or applications that may run on a variety of different hardware but all submit digital representations of biometrics and related requests in a uniform data structure format. In this way, a system that uses the biometrics to centrally (and/or virtually centrally in implementations where the system is implemented as using within a cloud network) control access to identity information may be configured to process the uniform data structure format to receive, extract, process, and respond to any digital representation of any biometrics and/or related requests regardless of the hardware used to obtain and/or transmit a digital representation of a biometric and/or a related request, the medical service provider who implements and/or uses such hardware, and so on. Further, such a uniform data structure format may allow the system to use different biometrics, different numbers of biometrics, and/or respond to different requests without reconfiguration of the system, client apps or applications, hardware used to obtain and/or transmit digital representations of biometrics and/or related requests, and so on.

In this way, systems described by the present disclosure may be able to check in people for a variety of different medical services at a variety of different medical service providers in a way that is not burdensome while being efficient. Additionally, such systems may be able to perform functions not possible by previous systems while reducing duplicated components, reducing excess processing, reducing excess communication network traffic, improving the efficiency of system hardware and software resources, reducing the number of personnel used to operate the system, and so on.

The following disclosure relates to a system for biometric secured medical check in. The system may receive one or more digital representations of biometrics for a person, use the digital representation of the biometric to retrieve identity information for the person, and provide the identity information to a medical service electronic device to check in the person for a medical service. In some implementations, the system may use the digital representation of the biometric to retrieve a medical record identifier for the person and facilitate access to a medical record for the person stored by a medical records electronic device. In various implementations, the system may process payment for the medical service using payment information stored in association with the identity information. In a number of implementations, the system may receive the digital representation of the biometric from a check in electronic device and provide an acknowledgement based on a response received from the medical service electronic device to the check in electronic device.

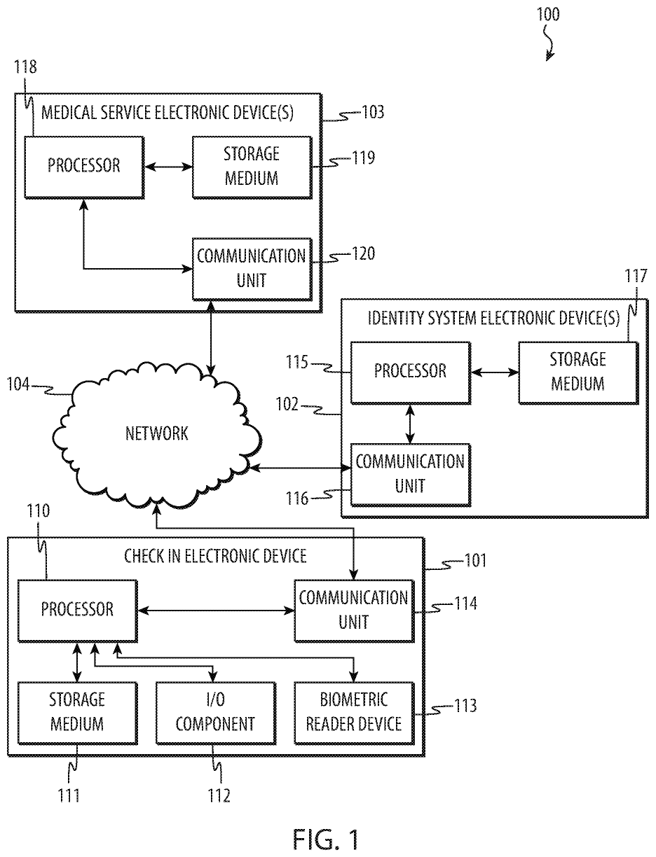

These and other embodiments are discussed below with reference to FIGS. 1-13. However, those skilled in the art will readily appreciate that the detailed description given herein with respect to these Figures is for explanatory purposes only and should not be construed as limiting.

FIG. 1 depicts a first example system 100 for biometric secured medical check in. The system 100 may include a check in electronic device 101, one or more identity system electronic devices 102, and/or one or more medical service electronic devices 103 that may be operative to communicate with each other via one or more communication networks 104.

The check in electronic device 101 may obtain one or more digital representations (which may be in the form of one or more hashes of an electronic representation of the biometric and/or other data structures) of one or more biometrics from a person. The check in electronic device 101 may provide the digital representation of the biometric to the identity system electronic device 102. Alternatively, the check in electronic device 101 may provide the digital representation of the biometric to the identity system electronic device 102 via the medical service electronic device 103. The identity system electronic device 102 may receive the digital representation of the biometric, use the digital representation of the biometric to retrieve one or more sets of identity information associated with the person, and provide the retrieved identity information to the medical service electronic device 103. The medical service electronic device 103 may receive the identity information and use the identity information to check in the person for a medical service.

For example, a person may provide a fingerprint, a facial image, and/or another biometric to the check in electronic device 101. The check in electronic device 101 may transmit a digital representation of the biometric to the identity system electronic device 102, which may use the digital representation of the biometric to retrieve a name and/or other patient identifier for the person and provide the name and/or other patient identifier for the person to the medical service electronic device 103. The medical service electronic device 103 may use the name to determine that the person has an appointment for a medical service and check in the person for that determined medical service.

In various implementations, the identity system electronic device 102 and/or the medical service electronic device 103 may provide one more responses and/or acknowledgements to the person via the check in electronic device 101. For example, the check in electronic device 101 may present one or more check in confirmations based on a received response and/or acknowledgment. Such a check in confirmation may include directions regarding where to go (such as a room number) for the medical service, instructions regarding preparation for the medical service (such as instructions regarding rolling up a sleeve in preparation for an inoculation), an estimated wait time, information regarding the medical service that is to be provided (such as a description of a procedure, information regarding a medical professional who will provide the medical service, and so on), information regarding future medical services to be provided and/or scheduled, and so on, costs associated with the medical service, and so on. By way of another example, the check in electronic device 101 may present one or more prompts based on a received response and/or acknowledgment. Such prompts may include a request for insurance information, payment account information, authorization to release identity information and/or medical records, directions regarding specific sets of identity information and/or medical records to release, selection of a medical service provider location, selection between a number of possible medical service appointments, medical waiver signature, acknowledgement of medical service risks, and so on. Alternatively, in other implementations, such check in confirmations, prompts, and so on may be transmitted to an electronic device associated with the person instead of the check in electronic device 101.

In some implementations, the identity system electronic device 102 may determine the medical service electronic device 103 to which to provide the identity information. For example, the identity system electronic device 102 may receive location information from the check in electronic device 101 (such as location information provided and/or selected by the person, included in an identifier provided by the check in electronic device 101, such as a network address, obtained via a global positioning system device, and so on) and determine the medical service electronic device 103 that corresponds to that location.

In various implementations, the identity system electronic device 102 may allow the person to control access to the identity information and/or other information (such as payment account information, medical records, Health Insurance Portability and Accountability Act protected information in order to be compliant with various legal restrictions, and so on). The identity system electronic device 102 may control access to such information according to input received from the person.

The system 100 may protect data by avoiding storing identity information and/or biometric data and/or other information at the medical service electronic device 103 and/or the check in electronic device 101. The system 100 may also protect data by using biometrics to control access to the devices that do store such data. In other implementations, the system 100 may perform other functions, such as charging insurance for medical services, charging a payment account for a medical service, communicating with a medical record database to facilitate secure access by the medical service electronic device 103 to medical records, providing expedited and/or discounted access to medical services and/or complimentary and/or other goods or services to premium account holders or reward account holders, contacting a person's preferred pharmacy to provide a prescription for the person, contacting pharmacies to evaluate costs and/or compare costs between a person's preferred pharmacy and at least one other pharmacy, offering a person to send a prescription to a cheaper pharmacy than the person's preferred pharmacy and/or informing the person of the cost savings, and so on. Various configurations are possible and contemplated without departing from the scope of the present disclosure.

The check in electronic device 101 may be any kind of device. The check in electronic device 101 may be provided by the medical service provider (such as a kiosk or other station in a reception area), may be a device associated with the person (such as the person's mobile telephone), and so on. Examples of such devices include, but are not limited to, one or more desktop computing devices, laptop computing devices, mobile computing devices, wearable devices, tablet computing devices, mobile telephones, smart phones, printers, displays, vehicles, kitchen appliances, entertainment system devices, digital media players, and so on. The check in electronic device 101 may include one or more processing units 110 and/or other processors and/or controllers, one or more non-transitory storage media 111 (which may take the form of, but is not limited to, a magnetic storage medium; optical storage medium; magneto-optical storage medium; read only memory; random access memory; erasable programmable memory; flash memory; and so on), one or more input/output components 112 (such as one or more displays, touch screens, printers, microphones, speakers, keyboards, computer mice, track pads, and so on), one or more biometric reader devices 113 (such as a fingerprint scanner, a vein scanner, a palm-vein scanner, an optical fingerprint scanner, a phosphorescent fingerprint scanner, a still image and/or video camera, a 2D and/or 3D image sensor, a capacitive sensor, a saliva sensor, a deoxyribonucleic acid sensor, a heart rhythm monitor, a microphone, and so on), one or more communication units 114, and/or one or more other components. The processing unit 110 may execute one or more sets of instructions stored in the non-transitory storage media 111 to perform various functions, such as using the biometric reader device 113 to obtain one or more digital representations of one or more biometrics (such as a digital representation of a fingerprint, a vein scan, a palm-vein scan, a voiceprint, a facial image, a retina image, an iris image, a deoxyribonucleic acid sequence, a heart rhythm, a gait, and so on) for a person, communicate with the identity system electronic device 102 and/or the medical service electronic device 103 via the network 104 using the communication unit 114, and so on.

Similarly, the identity system electronic device 102 may be any kind of electronic device and/or cloud and/or other computing arrangement and may include one more processing units 115, communication units 116, non-transitory storage media 117, and/or other components. The processing unit 115 may execute one or more sets of instructions stored in the non-transitory storage media 117 to perform various functions, such as storing biometric data for people and associated identity information (such as one or more names, addresses, telephone numbers, social security numbers, patient identification numbers or other identifiers, insurance data, financial data, medical history, and so on), receive one or more digital representations of biometrics, match one or more received digital representations of biometrics to stored biometric data, retrieve identity information associated with stored biometric data matching one or more received digital representations of biometrics, provide retrieved identity information, communicate with the check in electronic device 101 and/or the medical service electronic device 103 via the network 104 using the communication unit 116, and so on.

Likewise, the medical service electronic device 103 may be any kind of electronic device and/or cloud and/or other computing arrangement and may include one or more processing units 118, non-transitory storage media 119, communication units 120, and/or other components. The processing unit 118 may execute one or more sets of instructions stored in the non-transitory storage media 119 to perform various functions, such as store information regarding one or more medical services and/or appointments for one or more medical services, receive identity information, check in people for medical services using received identity information, communicate with the check in electronic device 101 and/or identity system electronic device 102 via the network 104 using the communication unit 120, and so on.

Although the system 100 is illustrated and described as including particular components arranged in a particular configuration that perform particular functions, it is understood that this is an example. In various implementations, various arrangements of various components that perform various functions may be implemented without departing from the scope of the present disclosure.

For example, in some implementations, the functions of the check in electronic device 101 may be performed using an app or application (such as an Internet browser) executing on a person's portable computing device (such as a smart phone) and the functions of the medical service electronic device 103 may be performed by a networked group of computing devices located at a medical service provider's location. However, in other implementations, a single electronic device or group of devices may perform the functions of both the check in electronic device 101 and the medical service electronic device 103. For example, a kiosk or other station located in the reception area of a medical service provider's office may be operative to receive digital representations of biometrics, transmit the digital representations of biometrics to the identity system electronic device 102, receive identity information, determine an appointment corresponding to information included in the identity information, check in a person for a determined appointment using the identity information, and/or various other functions. Various configurations are possible and contemplated without departing from the scope of the present disclosure.

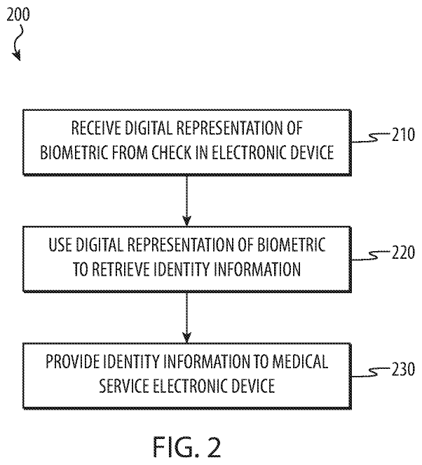

FIG. 2 depicts a flow chart illustrating a first example method 200 for biometric secured medical check in. This method 200 may be performed by the system 100 of FIG. 1.

At operation 210, an electronic device, such as the identity system electronic device 102, may receive a digital representation of a biometric. The electronic device may receive the digital representation of the biometric from a check in electronic device.

At operation 220, the electronic device may use the digital representation of the biometric to retrieve identity information. For example, the electronic device may match the digital representation of the biometric to stored biometric data and retrieve identity information (whether stored by the electronic device or another device) that is associated with matching stored biometric data.

At operation 230, the electronic device may provide the retrieved identity information to a medical service electronic device. For example, the electronic device may transmit a retrieved patient identification number and/or other patient identifier to a medical service electronic device to facilitate checking in a person for a medical service appointment.

In various examples, this example method 200 may be implemented as a group of interrelated software modules or components that perform various functions discussed herein. These software modules or components may be executed within a cloud network and/or by one or more computing devices, such as the identity system electronic device 102, medical service electronic device 103, and/or the check in electronic device 101 of FIG. 1.

Although the example method 200 is illustrated and described as including particular operations performed in a particular order, it is understood that this is an example. In various implementations, various orders of the same, similar, and/or different operations may be performed without departing from the scope of the present disclosure.

For example, the method 200 is illustrated and described as retrieving and providing identity information. However, in some implementations, only a subset of retrieved identity information may be provided. For example, the electronic device may retrieve more identity information than is requested and may only provide the requested identity information. In other examples, the electronic device may receive more identity information than the electronic device is authorized to provide and may only provide the authorized identity information. Various configurations are possible and contemplated without departing from the scope of the present disclosure.

In a number of implementations, a system for biometric secured medical check in may include at least one non-transitory storage medium that stores instructions and at least one processor. The at least one processor may execute the instructions to receive a digital representation of a biometric of a person from a check in electronic device, use the digital representation of the biometric to retrieve identity information for the person, check the person in for a medical service by providing the identity information to a medical service electronic device, receive a response from the medical service electronic device, and provide an acknowledgment based on the response to the check in electronic device.

In some examples, the acknowledgement may prompt for authorization to access a medical record for the person. In various examples, the acknowledgement may include an instruction regarding a location to report to receive the medical service. In a number of examples, the at least one processor may determine the medical service electronic device to provide the identity information using location information provided via the check in electronic device. In some examples, the at least one processor may determine the medical service electronic device to provide the identity information using a location of the check in electronic device. In various examples, the system may transmit a reminder prior to check in, such as a check in reminder corresponding to an appointment that is transmitted to a remote or other mobile device that enables a person to check in remotely ahead of time and then confirm check in upon arrival. Such a check in reminder may include a notification for a potential charge for a missed appointment, offer incentives for checking in ahead of time (such as a discounted copay in order to reward people in order for medical service providers to have a better idea what their schedule for the day will be, and so on.

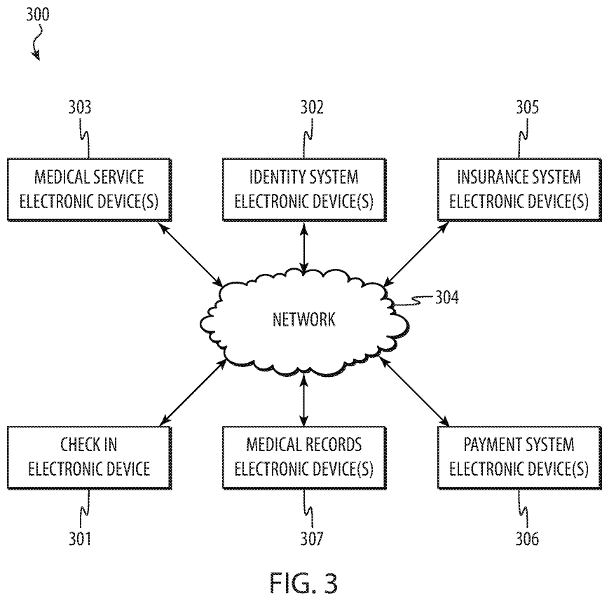

FIG. 3 depicts a second example system 300 for biometric secured medical check in. The system 300 may include a check in electronic device 301, one or more identity system electronic devices 302, one or more medical service electronic devices 303, one or more insurance system electronic devices 305 (such as an insurance claim system electronic device, an insurance coverage verification and/or information electronic device, and so on), one or more payment system electronic devices 306 (such as a credit card and/or other payment processing system electronic device), and/or one or more medical records electronic devices 307 (such as a database to securely store medical records and/or other electronic device) that may be operative to communicate with each other via one or more communication networks 304.

Similarly to the system 100 of FIG. 1, the identity system electronic device 302 may be operable to receive one or more digital representations of biometrics, use the digital representation of the biometric to retrieve identity information, and provide the identity information to the medical service electronic device 303 and/or one or more other electronic devices. Additionally, the identity system electronic device 302 may be operable to process payments and/or facilitate payment processing for one or more medical services.

For example, the identity system electronic device 302 may store insurance information included in and/or associated with the identity information. The identity system electronic device 302 may receive information from the medical service electronic device 303 regarding a medical service, the cost of a medical service, and so on; retrieve the insurance information; and communicate with the insurance system electronic device 305 to charge the insurance and/or otherwise process payment for the medical service. Alternatively, the identity system electronic device 302 may provide the insurance information to the medical service electronic device 303 and the medical service electronic device 303 may communicate with the insurance system electronic device 305 directly.

By way of another example, the identity system electronic device 302 may store financial information (such as one or more credit card numbers, health savings account numbers, flex spending account numbers, debit card numbers, checking or savings account numbers, and/or other financial account numbers, such as an airline mileage account or other rewards or loyalty account that may be used to make a payment) included in and/or associated with the identity information. The identity system electronic device 302 may receive information from the medical service electronic device 303 and/or the insurance system electronic device 305 regarding a medical service, the cost of a medical service, a copay or other payment amount a person is responsible for despite insurance coverage, and so on; retrieve the financial information; and communicate with the payment system electronic device 306 to process a payment, facilitate payment processing, obtain payment for a copay, charge a financial account number, and so on. Alternatively, the identity system electronic device 302 may provide the financial account number and/or other financial and/or payment information (such as insurance information) to the medical service electronic device 303 and the medical service electronic device 303 may communicate with the payment system electronic device 306 directly.

Moreover, the identity system electronic device 302 may be operable to facilitate and/or otherwise provide access to one or more medical records, such as those stored by the medical records electronic device 307. For example, the identity system electronic device 302 may store one or more medical record identifiers in and/or otherwise associated with the identity information. By way of another example, the medical record identifier may be based on the identity information, such as an implementation where the medical record identifier is a hash of at least a portion of the identity information (such as a hash of a digital representation of a biometric, a hash of a social security number or other identifier that can be used to uniquely identify a person without providing access to the identifier, a hash of a name and a social security number, and so on) coupled with an identifier for one of a number of different records repositories such that the medical record identifier is a unique medical record identifier enabling access into that specific records repository. In various examples, the identity information may include data enabling translation between a patient identifier used by an individual medical service provider and/or group of medical service providers and the medical record identifier. Regardless, the identity system electronic device 302 may be operative to retrieve the medical record identifier and use the medical record identifier to facilitate access by the medical service electronic device 303 to a medical record stored by the medical records electronic device 307. In this way, the identity system electronic device 302 may facilitate access to the medical records without storing the medical records.

For example, the identity system electronic device 302 may provide the medical record identifier and/or a specification of medical records requested to the medical records electronic device 307, receive and then provide one or more medical records (such as to the medical service electronic device 303) and/or direct where such medical records should be received, and so on. In some implementations, the identity system electronic device 302 may obtain authorization first, such as by communicating with the check in electronic device 301 and/or another device associated with the person to obtain authorization to access medical records, by referencing stored preferences regarding medical record access, and so on. By way of another example, the identity system electronic device 302 may provide the medical record identifier to another device (such as the medical service electronic device 303) that may then communicate with the medical records electronic device 307 directly.

The medical records electronic device 307 may centrally store medical records (and/or virtually centrally store in implementations where the medical records electronic device 307 is implemented as using within a cloud network) for a person from a variety of different medical service providers. As such, a person may not be required to remember and/or bring complex medical history information and/or go through burdensome and/or time consuming procedures to transfer medical records (such as when switching doctors). The medical records electronic device 307 may be configured to receive updates regarding provided medical services and/or other information to store in medical records (such as information from the medical service electronic device 303 regarding a medical service that is provided to a person), provide medical records in response to receiving a medical record identifier and/or legal authorization to provide medical records, push updates to associated authorized medical service providers, provide notifications to associated authorized medical service providers that updates are available for them to access, and so on. The medical records electronic device 307 may include one or more medical histories and/or portions thereof, allergies, vaccination lists, electronic health record data, electronic medical record data, patient chart data, Health Insurance Portability and Accountability Act and/or other consent forms, current medication lists, prescriptions, previous surgeries, previous hospitalizations, medical consents, upcoming medical service appointments, lab diagnostic results, lab imaging results, order for lab tests, and/or any other medical record information.

Additionally, in various implementations, the identity system electronic device 302 may be operable to provide one or more attestations regarding a person associated with the identity information. For example, a medical service may be restricted to people who are at least 21 years of age and/or who have parental consent. In such implementations, the identity system electronic device 302 may store a verified age for the person (such as in the identity information and/or associated therewith) and/or be operable to communicate with an age verification database. As such, the identity system electronic device 302 may be operable to verify the age of the person and provide one or more attestations regarding such to confirm that the person may legally be provided the medical service. Various configurations are possible and contemplated without departing from the scope of the present disclosure.

Moreover, in various implementations, the system 300 may control access using the identity information. For example, the system 300 may lock/unlock one or more rooms using the identity information, control access to one or more medications and/or information, and so on. Various configurations are possible and contemplated without departing from the scope of the present disclosure.

Although the system 300 is illustrated and described as including the insurance system electronic device 305, the payment system electronic device 306, and the medical records electronic device 307, it is understood that this is an example. In various implementations, one or more of these devices may be included without utilizing all. Various configurations are possible and contemplated without departing from the scope of the present disclosure.

Further, although the system 300 is described above as facilitating insurance and/or other payment processing and/or access to medical records contemporaneous with checking a person in for a medical service, it is understood that this is an example. In various implementations, the system 300 may perform these operations at different times (such as checking in a person upon arrival for a medical service, facilitating access to medical records during the medical service, verifying a person's identity after check in but before performance of a medical procedure to ensure that the medical procedure is performed on the same person who checked in, processing payment after the medical service, and so on). In some examples, the digital representation of the biometric and/or other digital representations of biometrics may be obtained for each operation, obtained once for an entire chain of operations, and so on. Various configurations are possible and contemplated without departing from the scope of the present disclosure.

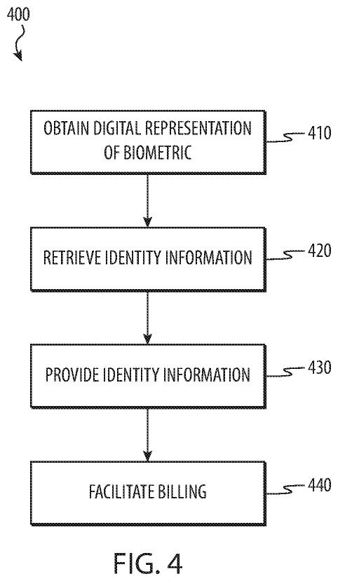

FIG. 4 depicts a flow chart illustrating a second example method 400 for biometric secured medical check in. This method 400 may be performed by the systems 100, 300 of FIGS. 1 and/or 3.

At operation 410, an electronic device (such as the identity system electronic device 102, 302 of FIGS. 1 and/or 3) may obtain a digital representation of a biometric. For example, the electronic device may receive the digital representation of the biometric from a biometric reader device, from another electronic device (such as the check in electronic device 101, 301 of FIGS. 1 and/or 3), and so on.

At operation 420, the electronic device may retrieve identity information using the digital representation of the biometric. The electronic device may retrieve the identity information from a storage component of the electronic device, from an external database, and so on.

At operation 430, the electronic device may provide the identity information. For example, the electronic device may provide the identity information to an electronic device operated by a medical service provider (such as the medical service electronic device 103, 303 of FIGS. 1 and/or 3), to an electronic device associated with a person such as the check in electronic device 101, 301 of FIGS. 1 and/or 3) for display, editing, and/or authorization to pass on the identity information to another electronic device, and so on. The electronic device may check in a person for a medical service and/or facilitate such as part of providing the identity information.

At operation 440, the electronic device may facilitate billing. For example, the identity information may include and/or be associated with payment information (such as insurance information, one or more financial account numbers, and so on). The electronic device may retrieve the payment information and use the payment information to process payment (such as processing an insurance or financial account number payment for a medical service), provide payment information to another device to schedule billing using the payment information, and so on.

In various examples, this example method 400 may be implemented as a group of interrelated software modules or components that perform various functions discussed herein. These software modules or components may be executed within a cloud network and/or by one or more computing devices, such as the identity system electronic devices 102, 302 and/or the check in electronic devices 101, 301 of FIGS. 1 and/or 3.

Although the example method 400 is illustrated and described as including particular operations performed in a particular order, it is understood that this is an example. In various implementations, various orders of the same, similar, and/or different operations may be performed without departing from the scope of the present disclosure.

For example, operation 440 is illustrated and described as facilitating billing. However, it is understood that this is an example. In some implementations, facilitating billing may be replaced and/or supplemented with processing one or more payments (such as where insurance is billed but a payment for a copay is processed). Various configurations are possible and contemplated without departing from the scope of the present disclosure.

In some implementations, a system for biometric secured medical check in may include at least one non-transitory storage medium that stores instructions and at least one processor. The at least one processor may execute the instructions to receive a digital representation of a biometric of a person, use the digital representation of the biometric to retrieve identity information for the person, provide the identity information to a medical service electronic device to check the person in for a medical service, and process payment for the medical service using payment information stored in association with the identity information.

In various examples, the payment information may include insurance information for the person. In some such examples, the at least one processor may process the payment by submitting an insurance payment request using the insurance information. In a number of such examples, the at least one processor may process the payment by providing the insurance information to the medical service electronic device. In various such examples, the at least one processor may determine a copay associated with the medical service and the insurance information and obtain a payment from the person for the copay.

In a number of examples, the payment information may include a financial account number. In some such examples, the at least one processor may process the payment by charging the financial account number. In various such examples, the at least one processor may process the payment by providing the financial account number to the medical service electronic device.

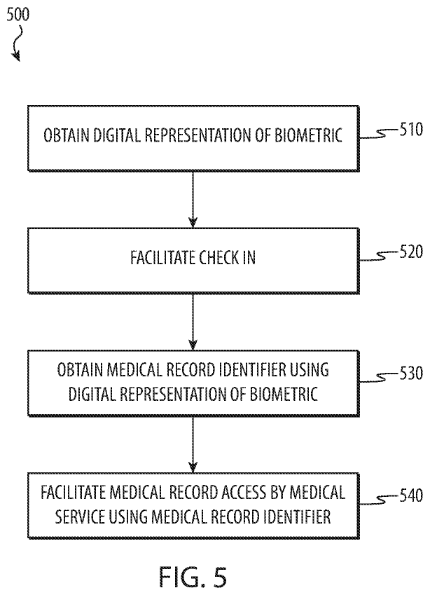

FIG. 5 depicts a flow chart illustrating a third example method 500 for biometric secured medical check in. This method 500 may be performed by the systems 100, 300 of FIGS. 1 and/or 3.

At operation 510, an electronic device (such as the identity system electronic device 102, 302 of FIGS. 1 and/or 3) may obtain a digital representation of a biometric. At operation 520, the electronic device may use the digital representation of the biometric to facilitate check in for a medical service.

For example, the electronic device may use the digital representation of the biometric to retrieve associated identity information. The electronic device may provide the identity information to an electronic device operated by a medical service provider (such as the medical service electronic device 103, 303 of FIGS. 1 and/or 3), which may use the identity information to check in a person for a medical service.

At operation 530, the electronic device may obtain a medical record identifier using the digital representation of the biometric. In some implementations, the medical record identifier may be included in and/or associated with the identity information.

At operation 540, the electronic device may facilitate medical record access by a medical service using a medical record identifier. For example, the electronic device may provide the medical record identifier and/or a specification of medical records requested to a medical records database (such as one that may be maintained by the medical records electronic device 307 of FIG. 3), receive and then provide one or more medical records to another electronic device (such as to the medical service electronic device 103, 303 of FIGS. 1 and/or 3) and/or direct where such medical records should be received, and so on. By way of another example, the electronic device may provide the medical record identifier to another device (such as the medical service electronic device 103, 303 of FIGS. 1 and/or 3) that may then communicate with the medical records database (such as one that may be maintained by the medical records electronic device 307 of FIG. 3) directly.

In various examples, this example method 500 may be implemented as a group of interrelated software modules or components that perform various functions discussed herein. These software modules or components may be executed within a cloud network and/or by one or more computing devices, such as the identity system electronic device 102, 302 and/or the check in electronic device 101, 301 of FIGS. 1 and/or 3.

Although the example method 500 is illustrated and described as including particular operations performed in a particular order, it is understood that this is an example. In various implementations, various orders of the same, similar, and/or different operations may be performed without departing from the scope of the present disclosure.

For example, the method 500 is illustrated and described as facilitating check in and medical records access in separate operations. However, in some implementations, facilitation of medical records access and check in may be part of a single operation. Various configurations are possible and contemplated without departing from the scope of the present disclosure.

In various implementations, a system for biometric secured medical check in may include at least one non-transitory storage medium that stores instructions and at least one processor. The at least one processor may execute the instructions to receive a digital representation of a biometric of a person, use the digital representation of the biometric to retrieve identity information for the person, provide the identity information to a medical service electronic device to check the person in for a medical service, use the digital representation of the biometric to retrieve a medical record identifier for the person, and use the medical record identifier to facilitate access by the medical service electronic device to a medical record for the person stored by a medical records electronic device.

In some examples, the at least one processor may facilitate the access by providing the medical record identifier to the medical service electronic device. In other examples, the at least one processor may facilitate the access by providing the medical record identifier to the medical records electronic device and providing a response from the medical records electronic device to the medical service electronic device.

In a number of examples, the medical record may include a vaccination list. In some examples, the medical record may include at least part of a medical history. In various examples, the medical record may include an allergy list. In a number of examples, the medical record may include a current medication list. In some examples, the medical record may include a preferred pharmacy. In various examples, the medical record may include a list of the person's current medical service providers. In some implementations, the list may include contact information for the person's current medical service providers so that one or more of the person's current medical service providers may be notified regarding medical services provided to the person. In a number of examples, the medical record may include a list of the person's past medical service providers. In some examples, the medical record may include a lab diagnostic result, a lab imaging result, an order for lab tests, and so on. Various configurations are possible and contemplated without departing from the scope of the present disclosure.

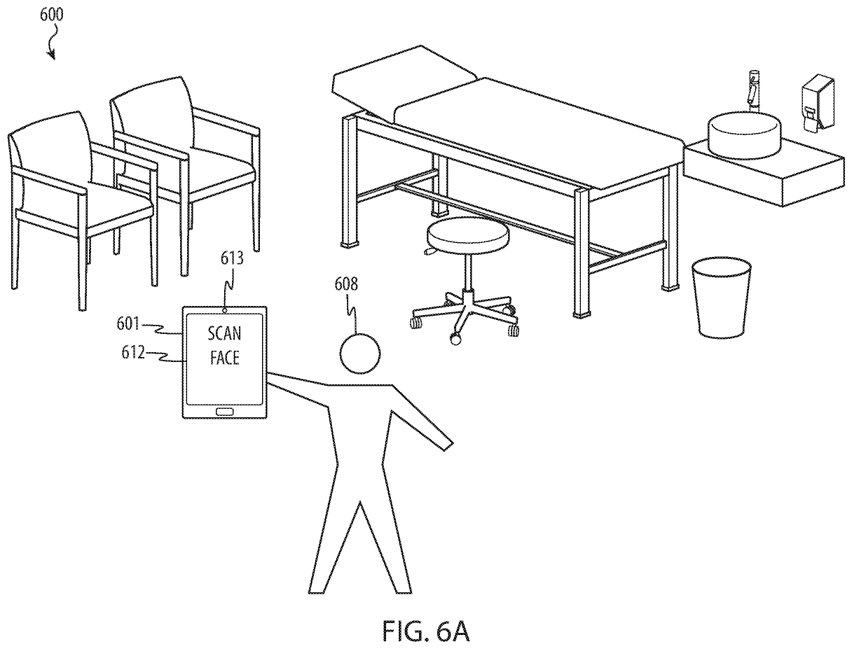

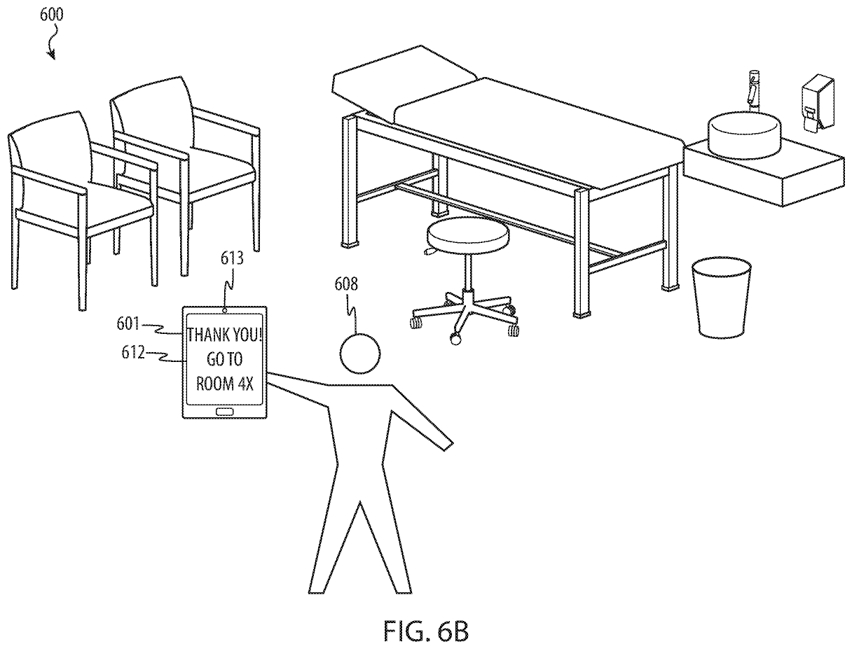

FIG. 6A depicts a third example system 600 for biometric secured medical check in. In this example, a person 608 may use a tablet computing device 601 in a doctor's waiting room. The tablet computing device 601 may include a 2D and/or 3D camera 613 and a screen 612. The tablet computing device 601 may execute an app and/or application that displays a message on the screen 612 prompting the person to scan an image of his face using the camera 613.

The person 608 may use the camera 613 to scan an image of his face. A digital representation of the image may be transmitted to an identity system. The identity system may receive the digital representation of the image, retrieve identity information using the digital representation of the image, and check in the person 608 for an appointment at the doctor's office (and/or facilitate such) by transmitting the identity information to a system at the doctor's office.

An acknowledgement of the check in may be transmitted to the tablet computing device 601, such as by the identity system, the system at the doctor's office, and so on. The acknowledgment may include instructions regarding the medical service. For example, as shown in FIG. 6B, the tablet computing device 601 may receive an acknowledgment and display such on the screen 612, indicating that the person 608 is to proceed to room 4X for the medical service.

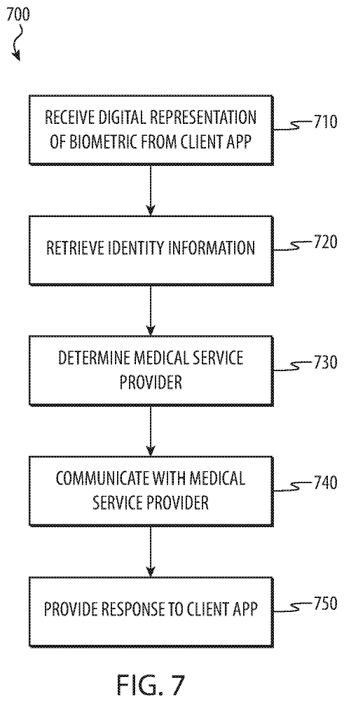

FIG. 7 depicts a flow chart illustrating a third example method 700 for biometric secured medical check in. This method 700 may be performed by the systems 100, 300, 600 of FIGS. 1, 3, and/or 6A and 6B.

At operation 710, an electronic device (such as the identity system electronic device 102, 302 of FIGS. 1 and/or 3) may receive a digital representation of a biometric from a client app and/or application, such as a client app and/or application executing on the tablet computing device 601 of FIGS. 6A and 6B. At operation 720, the electronic device may use the digital representation of the biometric to retrieve identity information. At operation 730, the electronic device may determine a medical service provider.

For example, the electronic device may determine a medical service provider according to an input received from a person via the client app and/or application. By way of another example, the electronic device may determine a medical service provider using a location component of an electronic device on which the client app and/or application is executing and comparing that location to medical service provider locations. In yet another example, the electronic device may determine a medical service provider according to a network via which the digital representation of the biometric was received and determining a medical service provider location associated with that network. In still another example, the electronic device may determine a medical service provider according to network identifiers included in a transmission associated with receipt of the digital representation of the biometric and determining a medical service provider location indicated by the network identifiers. In additional examples, the electronic device may determine a medical service provider based on data included in the identity information.

At operation 740, the electronic device may communicate with the determined medical service provider. For example, the electronic device may check in a person for a medical service and/or facilitate such by providing the identity information and/or a portion thereof to a system of the medical service provider.

The electronic device may receive one or more responses from the system, such as a confirmation of check in, an acknowledgement of the check in including instructions regarding the medical service, and so on. At operation 750, the electronic device may provide the response to the client app and/or application.

In various examples, this example method 700 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or the tablet computing device 601 of FIGS. 6A and 6B.

Although the example method 700 is illustrated and described as including particular operations performed in a particular order, it is understood that this is an example. In various implementations, various orders of the same, similar, and/or different operations may be performed without departing from the scope of the present disclosure.

For example, the method 700 is illustrated and described as providing a response to the client app and/or application. However, it is understood that this is an example. In some implementations, the response may be provided to another electronic device indicated in the identity information. In other implementations, no response may be provided. Various configurations are possible and contemplated without departing from the scope of the present disclosure.

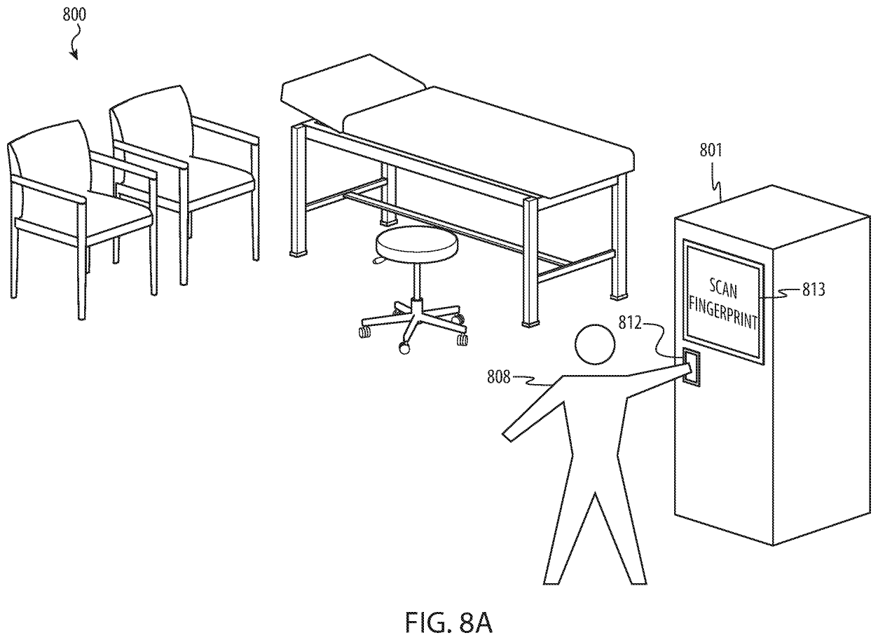

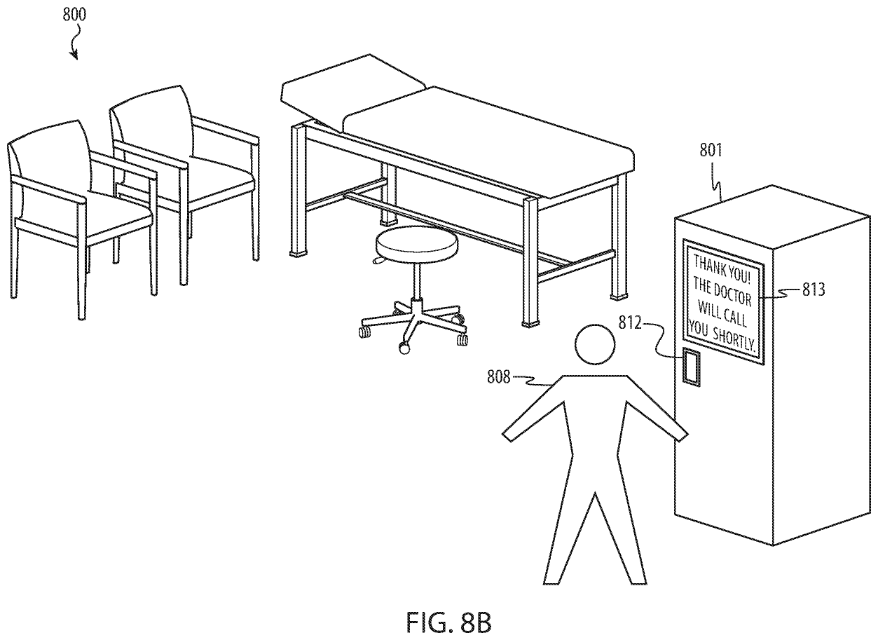

FIG. 8A depicts a fourth example system 800 for biometric secured medical check in. In this example, a person 808 may use a kiosk 801 or other station provided in a doctor's waiting room to check in for a medical service appointment. The kiosk 801 may include a fingerprint scanner 812 and a screen 813.

The kiosk 801 may execute an app and/or application that displays a message on the screen 813 prompting the person to scan his fingerprint using the fingerprint scanner 812. The person 808 may use the fingerprint scanner 812 to scan his fingerprint. A digital representation of the scan may be transmitted to an identity system. The identity system may receive the digital representation of the image, retrieve identity information using the digital representation of the image, and check in the person 808 for an appointment at the doctor's office (and/or facilitate such) by transmitting the identity information to a system at the doctor's office. An acknowledgement of the check in may be transmitted to the kiosk 801, such as by the identity system, the system at the doctor's office, and so on. For example, as shown in FIG. 8B, the kiosk 801 may receive an acknowledgment and display such on the screen 813, indicating that the doctor will call the person for the medical service shortly.

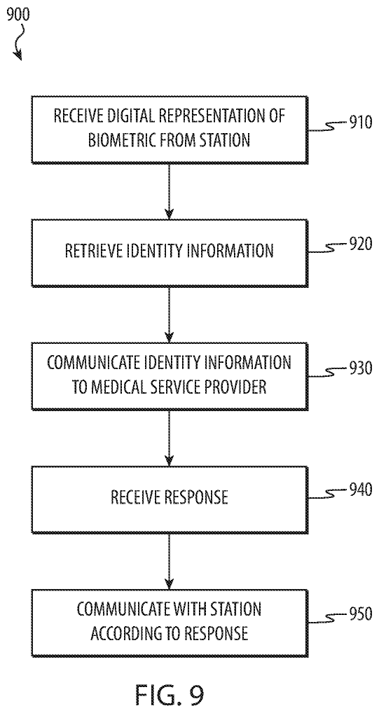

FIG. 9 depicts a flow chart illustrating a fourth example method 900 for biometric secured medical check in. This method 900 may be performed by the systems 100, 300, 800 of FIGS. 1, 3, and/or 8A and 8B.

At operation 910, an electronic device (such as the identity system electronic device 102, 302 of FIGS. 1 and/or 3) may receive a digital representation of a biometric from a station, such as the kiosk 801 of FIGS. 8A and 8B. At operation 920, the electronic device may use the digital representation of the biometric to retrieve identity information. At operation 930, the electronic device may communicate the identity information to a system of a medical service provider, such as the doctor's office at which the kiosk 801 of FIGS. 8A and 8B is located.

At operation 940, the electronic device may receive one or more responses from the system, such as a confirmation of a medical service check in, an acknowledgement of a medical service check in including instructions regarding the medical service, and so on. At operation 950, the electronic device may communicate with the station according to the response.

For example, the response may indicate to instruct the station to display information. As such, the electronic device may communicate with the station to display information. In other examples, the response may indicate to obtain additional information (such as one or more selections, payments, authorizations, and so on). As such, the electronic device may communicate with the station to obtain the additional information. Various configurations are possible and contemplated without departing from the scope of the present disclosure.

In various examples, this example method 900 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or the kiosk 801 of FIGS. 8A and 8B.

Although the example method 900 is illustrated and described as including particular operations performed in a particular order, it is understood that this is an example. In various implementations, various orders of the same, similar, and/or different operations may be performed without departing from the scope of the present disclosure.

For example, the method 900 is illustrated and described as having the electronic device communicate with the station according to the response. However, it is understood that this is an example. In some implementations, the electronic device may not communicate with the station according to the response. In various examples, the electronic device may instead communicate with another electronic device, such as an electronic device indicated in the identity information and/or otherwise associated with the identity information. Various configurations are possible and contemplated without departing from the scope of the present disclosure.

Although medical services are described numerous times above in the context of an appointment at a doctor's office, it is understood that this is an example. The present disclosure may be used in contexts other than medical services without departing from the scope of the present disclosure and medical services may be any kind of service provided in relation to medicine with or without a scheduled appointment. In some implementations, arriving at a pharmacy and/or other automated, partially automated, and/or non-automated medicine and/or medical product dispensary may constitute a check in for a medical service without departing from the scope of the present disclosure.

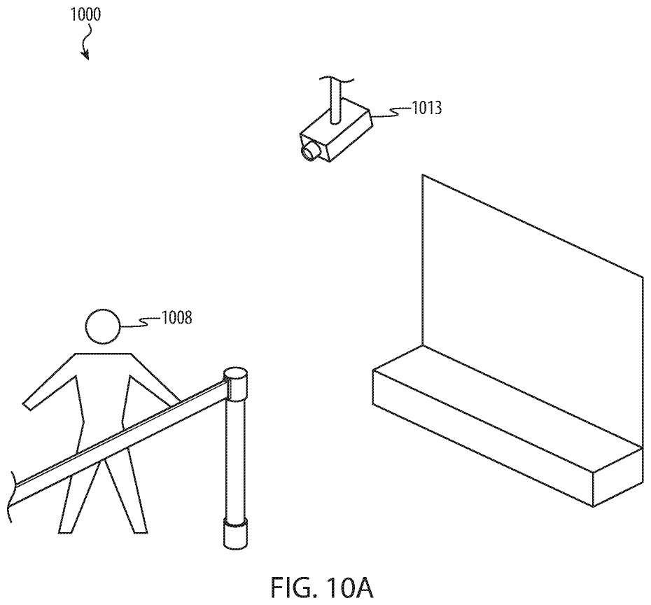

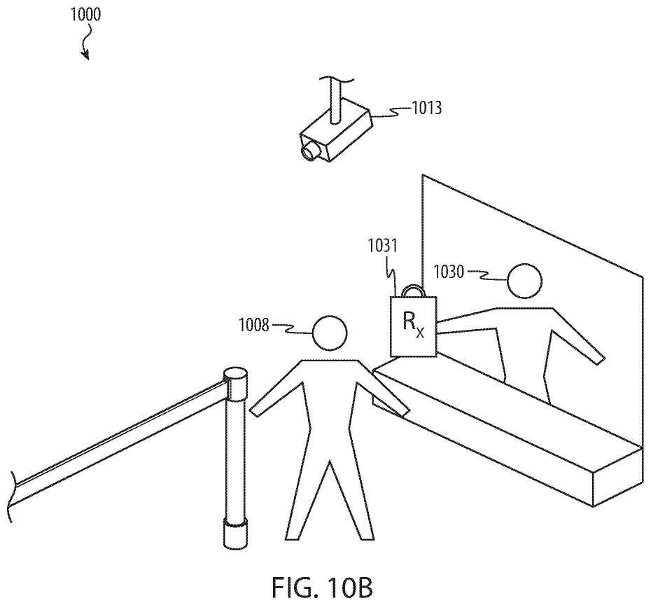

For example, FIG. 10A depicts a fifth example system 1000 for biometric secured medical check in. In this example, a person 1008 may wait in a line to approach a pharmacy counter. A camera 1013 may capture a digital representation of a face of the person 1008. Identity information for the person may be retrieved using the digital representation of the person's face. As shown in FIG. 10B, the identity information may be used to determine one or more medications 1031 to provide to the person 1008 and a pharmacist and/or other delivery mechanism may be directed to provide such.

For example, the identity information may be used to access medications 1031 that the person 1008 has requested. By way of another example, the identity information may be used to retrieve a medical record identifier for the person and access prescriptions indicated in a medical record accessible from a medical records system using the medical record identifier. In such an example, the medications 1031 may be selected using such prescriptions (such as a prescription that has been called into the pharmacy for the person 1008, a refill that the person 1008 has available and is due to pick up according to when a previous prescription fill would have been finished, and so on). In some examples, the identity information may be used to verify that the person is allowed to obtain the medication 1031, such as verifying an age of the person 1008 for prescriptions that may only legally be provided to people of a certain age (such as 18 years of age, 21 years of age, and so on), verifying that the person has not already obtained more than a regulated amount of a medical product (such as prescription cold medicines that may be restricted to a certain amount obtained in a single day, week, and so on), verifying that a prescription does not have an adverse interaction with another medication the person is indicated as taking in a medical record and/or the identity information, and so on.

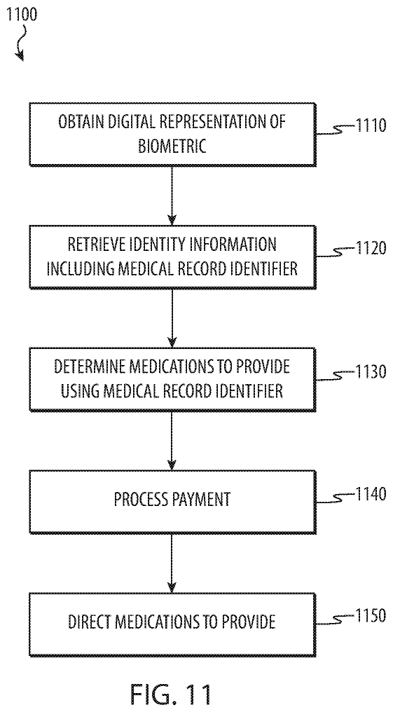

FIG. 11 depicts a flow chart illustrating a fifth example method 1100 for biometric secured medical check in. This method 1100 may be performed by the systems 100, 300, 1000 of FIGS. 1, 3, and/or 10A and 10B.

At operation 1110, an electronic device (such as the identity system electronic devices 102, 302 of FIGS. 1 and/or 3) located at a pharmacy and/or other medicine and/or medical product dispensing location may obtain a digital representation of a biometric for a person. At operation 1120, the electronic device may use the digital representation of the biometric to retrieve identity information. The identity information may include and/or be associated with a medical record identifier.

At operation 1130, the electronic device may determine one or more medications to provide using a medical record identifier. At operation 1140, the electronic device may process payment for the medications (such as using one or more credit card and/or other payment terminals, using payment information included in and/or associated with the identity information, and so on). At operation 1150, the electronic device may direct the medications to provide. For example, the electronic device may transmit and/or otherwise present a message to a pharmacist or other person regarding the medications to provide and/or the person to whom to provide the medications.

In various examples, this example method 1100 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic devices 101, 301 of FIGS. 1 and/or 3, and/or one or more electronic devices of the system 1000 of FIGS. 10A and 10B.

Although the example method 1100 is illustrated and described as including particular operations performed in a particular order, it is understood that this is an example. In various implementations, various orders of the same, similar, and/or different operations may be performed without departing from the scope of the present disclosure.

For example, the method 1100 is illustrated and described as processing payment. However, in some examples, the method 1100 may instead facilitate payment processing, omit payment processing, and/or perform other actions. Various configurations are possible and contemplated without departing from the scope of the present disclosure.

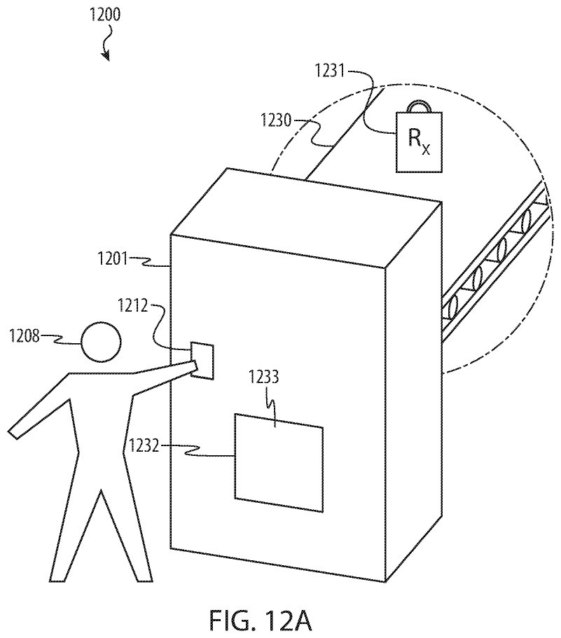

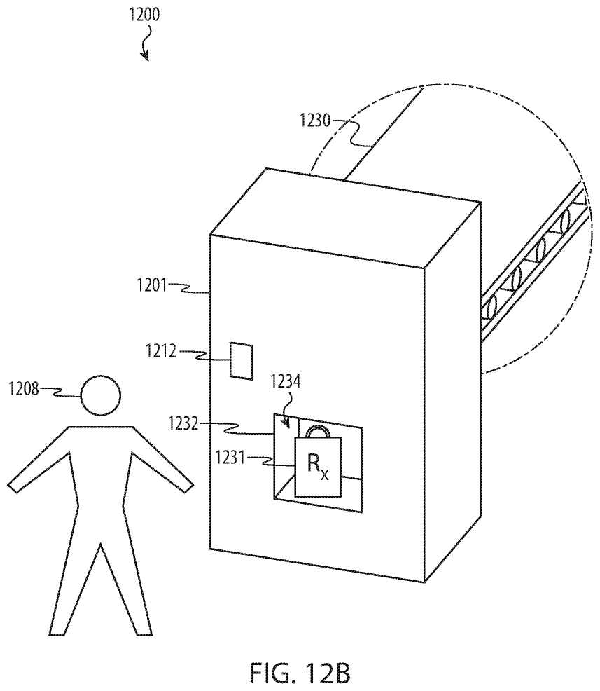

FIG. 12A depicts a sixth example system 1200 for biometric secured medical check in. In this example, a person 1208 may use a medical product automated dispensing device 1201 (such as a vending machine) to obtain one or more medical products 1231. The person 1208 may specify requested medical products and/or medical products 1231 may be determined for the person 1208.

The medical product automated dispensing device 1201 may include a fingerprint pad 1212. The person 1208 may use the fingerprint pad 1212 to provide one or more fingerprint images. The medical product automated dispensing device 1201 may obtain a digital representation of the fingerprint image, use such to retrieve identity information for the person, determine one or more medical products to dispense, obtain such medical products using one or more transport mechanisms, and provide the medical products.

For example, in this example, the medical product automated dispensing device 1201 may include a hatch 1232 that is covered by a door 1233. The medical product automated dispensing device 1201 may be connected to a medical product storage area via a conveyor belt 1230 and/or other delivery system hidden from the person 1208 on the other side of a wall. The medical product automated dispensing device 1201 may be configured to receive the medical products 1231 into the area of the hatch 1232 blocked by the door 1233 via the conveyor belt 1230. As shown in FIG. 12B, the medical product automated dispensing device 1201 may then withdraw the door 1233 to expose an aperture 1234 of the hatch 1232 where the medical products 1231 are located so that the person 1208 has access to the medical products 1231.

Although the above describes the medical product automated dispensing device 1201 as determining one or more medical products to dispense, it is understood that this is an example. In some implementations, the medical product automated dispensing device 1201 may provide the person 1208 a list of such medical products and allow the person 1208 to select among the list. The medical product automated dispensing device 1201 may then obtain and provide the selected metical products. Various configurations are possible and contemplated without departing from the scope of the present disclosure.

Further, although the above illustrates and describes a conveyor belt 1230, it is understood that this is an example and that other delivery systems may be used without departing from the scope of the present disclosure. For example, one or more rotating coils may be used to move one or more medical products from a shelf such that the medical product falls into an aperture that is accessible by the person 1208. Various configurations are possible and contemplated without departing from the scope of the present disclosure.

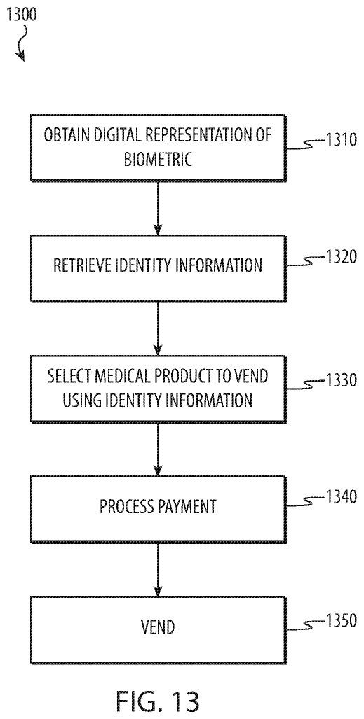

FIG. 13 depicts a flow chart illustrating a fifth example method 1300 for biometric secured medical check in. This method 1300 may be performed by the systems of FIGS. 1, 3, and/or 12A and 12B.

At operation 1310, an electronic device (such as the identity system electronic device 102, 302 of FIGS. 1 and/or 3 and/or the medical product automated dispensing device 1201 of FIG. 12) may obtain a digital representation of a biometric. At operation 1320, the electronic device may use the digital representation of the biometric to retrieve associated identity information. At operation 1330, the electronic device may use the identity information to select one or more medical products to vend.

At operation 1340, the electronic device may process one or more payments for the medical product. For example, the electronic device may include a credit/debit card reader, a bill collector, and so on and may use such to process payment. By way of another example, the electronic device may use payment information associated with the identity information to process payment. At operation 1350, the electronic device may vend the medical product.

In various examples, this example method 1300 may be implemented as a group of interrelated software modules or components that perform various functions discussed herein. These software modules or components may be executed within a cloud network and/or by one or more computing devices, such as the identity system electronic devices 102, 302 of FIGS. 1 and/or 3, the check in electronic device 101, 301 of FIGS. 1 and/or 3, and/or one or more electronic devices of the system 1200 of FIGS. 12A and 12B.

Although the example method 1300 is illustrated and described as including particular operations performed in a particular order, it is understood that this is an example. In various implementations, various orders of the same, similar, and/or different operations may be performed without departing from the scope of the present disclosure.

Although the method 1300 is illustrated and described in the context of a medical product vending machine, it is understood that this is an example. In various implementations, a variety of automated and/or semi-automated medical product providing systems other than vending machines may perform the method 1300. Various configurations are possible and contemplated without departing from the scope of the present disclosure.

Although the above is illustrated and described within the context of biometric secured medical check in, it is understood that this is an example. In various implementations, the systems of FIGS. 1, 3, and/or 12A and 12B and/or other techniques discussed herein may be used in a variety of contexts without departing from the scope of the present disclosure.

For example, identity systems (identity system electronic devices 102, 302 of FIGS. 1 and/or 3) may be used to control access to identity information (such as using digital images of one or more fingerprints, irises, faces, and/or other biometrics to identify people, authenticate identity, access related identity information, and so on) in order to facilitate a variety of functions. The identity system may interact with one or more electronic devices in order to determine and/or otherwise verify and/or authenticate a person's identity, validate a driver's license or other identity token for a person or provide information therefrom, validate an insurance card for a person or provide information thereof or function as a replacement for such, process payments using one or more credit cards and/or other financial accounts and/or provide information therefrom, validate one or more credit cards and/or other financial accounts and/or verify authorization to use such, verify boarding pass and/or other ticketing information (such as plane, bus, or train tickets; tickets to enter sporting or other venues; and so on), enable picking up of a rental vehicle, process payment for goods or services such as food and drinks, determine access to buildings, rooms, and/or other locations, and so on.

In various implementations, the identity system may interact with one or more electronic devices in order to perform various actions for patients whose identity information is accessible to the identity system. For example, the identity system may perform a variety of identification functions, such as positively identifying patients with confidence at one or more stages of their healthcare journey. By way of another example, the identity system may perform a variety of security functions, such as improving security and reducing fraud while minimizing and/or otherwise reducing cumbersome security protocols. In yet another example, the identity system may perform a variety of patient experience functions, such as enabling a seamless visit that focuses patients and staff on care rather than paperwork.

The identity system may connect the patient journey with an obtained digital representation of a biometric, such as a digital representation of a glance. The identity system may streamline the patient experience across the healthcare ecosystem and beyond with a unified biometric patient identifier, visits using biometrics or biometrics along with another identifier (such as a password, a physical item such as a card, and so on), and/or secure payments. The identity system may enable patients to check into an emergency room and/or other medical provider location (such as by validating identity, providing access to medical records and/or insurance, and so on), visit one or more labs for testing (which may ensure accuracy, reduce duplicate testing, and so on), be discharged to a specialist (which may involve enabling the patient to pay for a visit, receive instructions, and so on), receive services on arrival for an appointment (such as enabling self-service, paperless check in, verification, payment, and so on), pick up one or more prescriptions (in some examples allowing a prescription to be automatically and/or semi-automatically dispensed in response to a received digital representation of a biometric), share visit information with authorized physicians and/or other medical service providers, and so on.

In some implementations, the identity system may be used to reimagine the pharmacy experience. People may be able to safely access medication anywhere at any time. The identity system may streamline the person's experience by increasing access to controlled and/or prescription medication, in the store and/or beyond.

The identity system may enable innovation for the in-store medical product experience. The identity system may enable 24/7 medication pickup, which may reduce staffing costs and/or improve patient convenience at clinics and/or pharmacies. Integrated storage lockers and/or other devices may enable remote ordering (such as online, by phone, by text message, and so on) and pickup using digital representations of biometrics. The identity system may enable secure access to controlled substances and/or sensitive areas, simplify staff workflows, mitigate risk with better access, and oversight, and so on.

The identity system may enable pharmacies and/or other medical product providers to grow their retail footprint. For example, automated dispensing apparatuses (such as vending machines and so on) may be used to dispense behind-the-counter products and over-the-counter products at airports, stadiums, and/or other locations. The identity system may provide new ways to reach customers, such as via pharmacy delivery, dispensing solutions, and so on. The identity system may also enable embedded loyalty programs, which may drive behavioral change by incentivizing patients to stay healthy and adhere to health programs.

In various implementations, the identity system may enable reimagining of medical service provider employee experiences. The identity system may enable medical service provider employees a less burdensome and more secure way to go about their workday. This may boost employee satisfaction and/or data security across the healthcare ecosystem with a unified biometric identifier, access to rooms and/or other locations using biometrics or biometrics along with another identifier (such as a password, a physical item such as a card, and so on), workstations, substances, and so on. For example, the identity system may enable access of authorized personnel to a room, locker, or other storage area using biometrics or biometrics along with another identifier (such as a password, a physical item such as a card, and so on) where items such as prescription and/or other medications may be stored and may log who obtains access and/or any items accessed and/or removed. The identity system may integrate existing medical provider systems to reduce redundant tasks, such as by the identity system integrating information between a scheduling system and a billing system so that staff does not need to obtain patient information from the patient (and/or the scheduling system) that is already in the scheduling system in order to enter the information into the billing system.

For example, the identity system may enable an employee to walk into a clinic or other medical location without an identification card, access specialty areas (which may improve physical security without adding additional hassle), log into computers and/or other equipment (such may enable employees to spend less time accessing critical and/or other data), pay for goods or services (such as coffee, cafeteria food, and so on) without providing cash or cards (which may enable employees to enjoy breaks without holdups), access medication carts or other areas (which may decentralize access of controlled substances), head to hospitals or other locations for patient visits (which may remove excess access checkpoints), visit a lab for patient results (which may control contamination risks, patient results visibility, and so on), and so on. Various configurations are possible and contemplated without departing from the scope of the present disclosure.

As described above and illustrated in the accompanying figures, the present disclosure relates to a system for biometric secured medical check in. The system may receive one or more digital representations of biometrics for a person, use the digital representation of the biometric to retrieve identity information for the person, and provide the identity information to a medical service electronic device to check in the person for a medical service. In some implementations, the system may use the digital representation of the biometric to retrieve a medical record identifier for the person and facilitate access to a medical record for the person stored by a medical records electronic device. In various implementations, the system may process payment for the medical service using payment information stored in association with the identity information. In a number of implementations, the system may receive the digital representation of the biometric from a check in electronic device and provide an acknowledgement based on a response received from the medical service electronic device to the check in electronic device.

Although the above illustrates and describes a number of embodiments, it is understood that these are examples. In various implementations, various techniques of individual embodiments may be combined without departing from the scope of the present disclosure.

As described above and illustrated in the accompanying figures, the present disclosure relates to a system for authorizing a mobile identity information controlled device. At least one digital representation of a biometric may be received using a biometric reader device. Identity information may be obtained from an identity system device using the digital representation of the biometric. Operation of a mobile identity information controlled device may be controlled using the identity information. In this way, operation of a mobile identity information controlled device may be controlled using identity information while protecting access to the identity information. This may enable performance of functions not previously performable by the system, reduce the number of system components, prevent duplication of components, prevent identity information and/or biometric data from being stored by the mobile identity information controlled device, minimize communication connection traffic, improve the efficiency and/or operation of the system, and so on.

The present disclosure recognizes that biometric and/or other personal data is owned by the person from whom such biometric and/or other personal data is derived. This data can be used to the benefit of those people. For example, biometric data may be used to conveniently and reliably identify and/or authenticate the identity of people, access securely stored financial and/or other information associated with the biometric data, and so on. This may allow people to avoid repeatedly providing physical identification and/or other information.

The present disclosure further recognizes that the entities who collect, analyze, store, and/or otherwise use such biometric and/or other personal data should comply with well-established privacy policies and/or privacy practices. Particularly, such entities should implement and consistently use privacy policies and practices that are generally recognized as meeting or exceeding industry or governmental requirements for maintaining security and privately maintaining biometric and/or other personal data, including the use of encryption and security methods that meets or exceeds industry or government standards. For example, biometric and/or other personal data should be collected for legitimate and reasonable uses and not shared or sold outside of those legitimate uses. Further, such collection should occur only after receiving the informed consent. Additionally, such entities should take any needed steps for safeguarding and securing access to such biometric and/or other personal data and ensuring that others with access to the biometric and/or other personal data adhere to the same privacy policies and practices. Further, such entities should certify their adherence to widely accepted privacy policies and practices by subjecting themselves to appropriate third party evaluation.

Additionally, the present disclosure recognizes that people may block the use of, storage of, and/or access to biometric and/or other personal data. Entities who typically collect, analyze, store, and/or otherwise use such biometric and/or other personal data should implement and consistently prevent any collection, analysis, storage, and/or other use of any biometric and/or other personal data blocked by the person from whom such biometric and/or other personal data is derived.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A system for biometric secured medical check in, comprising:
at least one non-transitory storage medium that stores instructions; and
at least one processor that executes the instructions to:
receive a digital representation of a biometric of a person;
use the digital representation of the biometric to retrieve identity information for the person;
provide the identity information to a medical service electronic device to check the person in for a medical service;
use the digital representation of the biometric to retrieve a medical record identifier for the person; and
use the medical record identifier to facilitate access by the medical service electronic device to a medical record for the person stored by a medical records electronic device.

2. The system of claim 1, wherein the at least one processor facilitates the access by providing the medical record identifier to the medical service electronic device.

3. The system of claim 1, wherein the at least one processor facilitates the access by:
providing the medical record identifier to the medical records electronic device; and
providing a response from the medical records electronic device to the medical service electronic device.

4. The system of claim 1, wherein the medical record includes a vaccination list.

5. The system of claim 1, wherein the medical record includes at least part of a medical history.

6. The system of claim 1, wherein the medical record includes an allergy list.

7. The system of claim 1, wherein the medical record includes a current medication list.

8. A system for biometric secured medical check in, comprising:
at least one non-transitory storage medium that stores instructions; and
at least one processor that executes the instructions to:
receive a digital representation of a biometric of a person;
use the digital representation of the biometric to retrieve identity information for the person;
provide the identity information to a medical service electronic device to check the person in for a medical service; and
process payment for the medical service using payment information stored in association with the identity information.

9. The system of claim 8, wherein the payment information includes insurance information for the person.

10. The system of claim 9, wherein the at least one processor processes the payment by submitting an insurance payment request using the insurance information.

11. The system of claim 9, wherein the at least one processor processes the payment by providing the insurance information to the medical service electronic device.

12. The system of claim 9, wherein the at least one processor:
determines a copay associated with the medical service and the insurance information; and
obtains the payment from the person for the copay.

13. The system of claim 8, wherein the payment information includes a financial account number.

14. The system of claim 13, wherein the at least one processor processes the payment by charging the financial account number.

15. The system of claim 13, wherein the at least one processor processes the payment by providing the financial account number to the medical service electronic device.

16. A system for biometric secured medical check in, comprising:
at least one non-transitory storage medium that stores instructions; and
at least one processor that executes the instructions to:
receive a digital representation of a biometric of a person from a check in electronic device;
use the digital representation of the biometric to retrieve identity information for the person;
check the person in for a medical service by providing the identity information to a medical service electronic device;
receive a response from the medical service electronic device; and
provide an acknowledgment based on the response to the check in electronic device.

17. The system of claim 16, wherein the acknowledgement prompts for authorization to access a medical record for the person.

18. The system of claim 16, wherein the acknowledgement includes an instruction regarding a location to report to receive the medical service.

19. The system of claim 16, wherein the at least one processor determines the medical service electronic device to provide the identity information using location information provided via the check in electronic device.

20. The system of claim 16, wherein the at least one processor determines the medical service electronic device to provide the identity information using a location of the check in electronic device.
